(19) United States
(12) Patent Application Publication
(10) Pub. No.: US 2016/0238613 A1
(43) Pub. Date: Aug. 18, 2016

(54) ANTIGEN RECEPTOR SCREENING ASSAY

(71) Applicant: X-BODY, INC., Waltham, MA (US)

(72) Inventor: Richard W. Wagner, Waltham, MA (US)

(21) Appl. No.: 15/025,670

(22) PCT Filed: Sep. 26, 2014
(86) PCT No.: PCT/US2014/057672

§ 371 Date: Mar. 29, 2016

Related U.S. Application Data
(60) Provisional application No. 61/884,348, filed on Sep. 30, 2013.

(51) Int. Cl. G01N 33/68; G01N 21/78; C07K 16/28

(52) U.S. Cl. CPC G01N 33/6845; G01N 33/6854; C07K 16/2845; C07K 16/2821; G01N 21/78; G01N 2333/70553; G01N 2333/70525; C07K 2317/565; C07K 2317/567; C07K 2317/54; C07K 2317/21; G01N 2021/7773

(57) ABSTRACT

The present invention provides methods for the identification of an antigen receptor (e.g., an antibody) that specifically binds to an antigen of interest. Generally, this involves contacting a plurality of antigen receptor-expressing cells with an antigen of interest; measuring the level of activated adhesion molecules on the surface of the antigen receptor-expressing cells; and, identifying from the plurality of antigen receptor-expressing cells an antigen receptor-expressing cell that exhibits an increased amount of activated adhesion molecules on the cell surface.

### RELATED APPLICATION

This patent application claims priority from U.S. Provisional Patent Application No. 61/884,348, filed Sep. 30, 2013, the content of which is hereby incorporated by reference herein in its entirety.

### SUMMARY OF THE INVENTION

The present invention provides methods for the identification of an antigen receptor (e.g., an antibody) that specifically binds to an antigen of interest. The invention is based, at least in part, on the discovery that the binding of an antigen to a cognate antigen receptor on the surface of an antigen receptor-expressing cell (e.g., a B-cell) results in the activation of adhesion molecules (e.g., integrins) on the surface of that cell. These activated adhesion molecules are readily detectable (e.g.,, using label-free assay systems) and thereby serve to identify an antigen receptor-expressing cell comprising an antigen receptor that specifically binds to an antigen of interest. The methods of the invention are particularly useful for the identification of antibody-expressing cells (and the antibodies expressed therein) that specifically bind to an antigen of interest.

Accordingly, in one aspect the invention provides a method for identifying an antigen receptor that specifically binds to an antigen of interest, the method comprising: contacting a plurality of antigen receptor-expressing cells with the antigen; measuring the amount of activated adhesion molecules on the surface of the antigen receptor-expressing cells in the presence and absence of the antigen; and identifying from the plurality of antigen receptor-expressing cells an antigen receptor-expressing cell that specifically binds to the antigen, wherein an increase in the amount of activated adhesion molecules on the surface of an antigen receptor-expressing cell in the presence of the antigen relative to a suitable control is indicative of the binding of the antigen to the antigen receptor-expressing cell, thereby identifying an antigen receptor that specifically binds to an antigen of interest.

In certain embodiments, the method further comprises clonally isolating the identified antigen receptor-expressing cell.

In other embodiments, the method further comprises determining the nucleic acid or amino acid sequence of at least a portion of the identified antigen receptor.

In certain embodiments, the adhesion molecules are integrins. Suitable integrins include, without limitation, a Leukocyte Functional Antigen 1 (LFA-1) or Very Late Antigen 4 (VLA-4) molecule.

In certain embodiments, the amount of activated adhesion molecules is measured by measuring the binding of the antigen receptor-expressing cells to an extracellular matrix protein or to an antibody that binds to activated adhesion molecules but not to quiescent adhesion molecules. Suitable extracellular matrix proteins include, without limitation, an Inter-Cellular Adhesion Molecule 1 (ICAM-1) or a fibronectin molecule.

In certain embodiments, the binding of the antigen receptor-expressing cells to the extracellular matrix protein or the antibody is measured using a label-free biosensor coated with the extracellular matrix protein or the antibody. In a particular embodiment, the biosensor is a colorimetric resonant reflectance optical biosensor.

In certain embodiments, the antigen receptor is a B-cell receptor (e.g., a human B-cell receptor).

In certain embodiments, the antigen receptor-expressing cells are B-cells (e.g., human B-cells) or hybridoma cells. In a particular embodiment, the B-cells are isolated from one or more naïve animals (e.g., a human). In another particular embodiment, the B-cells are isolated from one or more animals (e.g., a human) that have not been immunologically challenged with the antigen of interest.

In certain embodiments, the antigen receptor-expressing cells (e.g., B-cells) have been immortalized.

In certain embodiments, the antigen receptor-expressing cells (e.g., B-cells) express endogenous antibodies.

In certain embodiments, the antigen receptor-expressing cells (e.g., B-cells) express a library of heterologous antibodies. In a particular embodiment, the library comprises a natural repertoire of unique antibodies (e.g., human antibodies). In another particular embodiment, the library is a naïve antibody library. In another particular embodiment, the library comprises a plurality of unique synthetic antigen receptors.

In certain embodiments, the antigen receptor-expressing cells (e.g., B-cells) express a library of unique chimeric antigen receptors, wherein each chimeric receptor comprises a portion of an antigen receptor (e.g., an antibody) linked to a heterologous binding molecule.

In another aspect the invention provides, a method for producing an antigen receptor (e.g., an antibody (e.g., a human antibody)) that specifically binds to an antigen of interest, the method comprising: identifying an antigen receptor according to the method of any one of the preceding claims; and expressing the antigen receptor, or an antigen-binding portion thereof.

In certain embodiments, the method further comprises determining the nucleic acid or amino acid sequence of at least one complementarity determining region (CDR) of the antibody. In a particular embodiment, the method further comprises grafting the at least one CDR into the framework of a heterologous antibody.

### DESCRIPTION OF THE FIGURES

FIG. 1 depicts a schematic of the “inside-out” signaling pathway that links B-cell receptor activation to integrin activation.

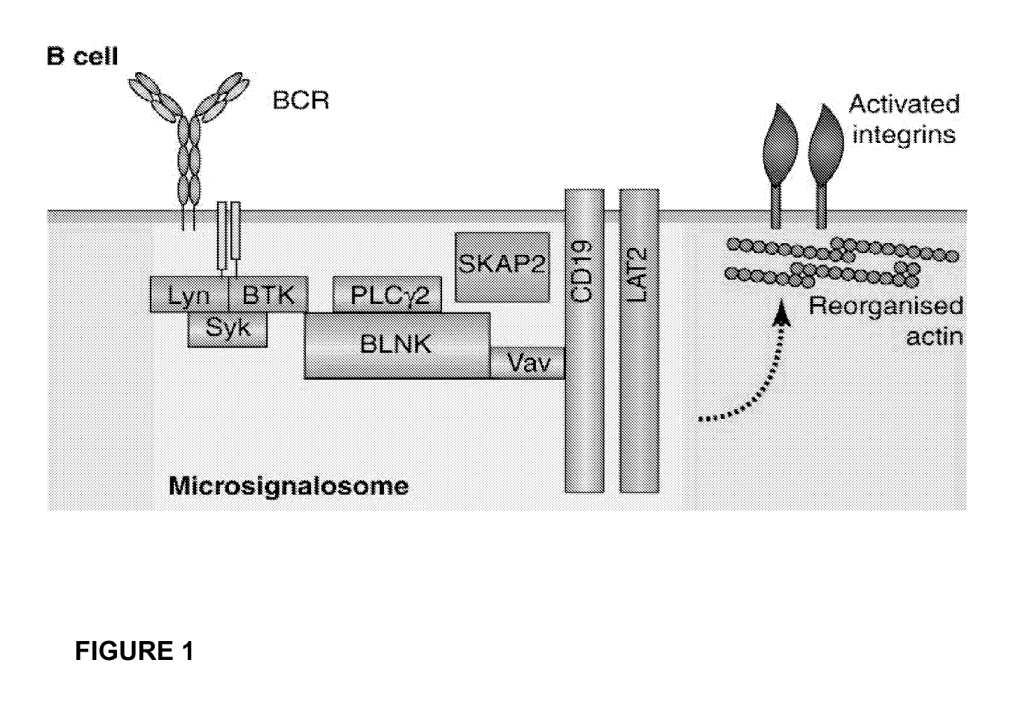

FIG. 2 depicts the results of label free biosensor assays measuring the binding of B-cells to extracellular matrix protein in response to B-cell receptor activation.

### DETAILED DESCRIPTION

The present invention provides methods for the identification of an antigen receptor that specifically binds to an antigen of interest. The methods of the invention generally involve contacting a plurality of antigen receptor-expressing cells with an antigen of interest; measuring the amount of activated adhesion molecules on the surface of the antigen receptor-expressing cells; and, identifying from the plurality of antigen receptor-expressing cells an antigen receptor-expressing cell that exhibits an increased amount of activated adhesion molecules on the cell surface.

### I. Definitions

As used herein, the term “antigen receptor” refers to the membrane bound antibody component of a B-cell receptor (i.e. a membrane bound antibody) or to the αβ chain or γδ chain components of a T-cell receptor. The term also encompasses chimeric antigen receptors wherein a portion of a B-cell or T-cell receptor has been replaced by heterologous binding molecules.

As used herein, the term “antibody” refers to IgG, IgM, IgA, IgD or IgE or an antigen-binding fragment thereof (e.g. VH and/or VL), whether derived from any species naturally producing an antibody, or created by recombinant DNA technology.

As used herein, the term “antigen” refers to a molecule that is recognized by an antigen receptor.

As used herein, the term “adhesion molecule” refers to a cell surface protein that mediates cell-cell or cell-extracellular matrix binding. An “activated” adhesion molecule is an adhesion molecule that has adopted a tertiary structure conformation that allows binding to cognate binding partners. A “quiescent” adhesion molecule is an adhesion molecule that has adopted a tertiary structure conformation that precludes binding to cognate binding partners.

As used herein, the term “suitable control” refers to any sample or reference value useful for identifying an increase in the amount of activated adhesion molecules on the surface of a cell. Suitable control samples include, without limitation, cells in the absence of antigen of interest. Suitable reference values include, without limitation, the average amount of activated adhesion molecules found on the surface of a cell in the absence of an antigen of interest.

As used herein, the term “specifically binds to” or “binds specifically” refers to the ability of an antigen receptor to bind to an antigen with an affinity of at least about 1×10⁻⁶ M, 1×10⁻⁷ M, 1×10⁻⁸ M, 1×10⁻⁹ M, 1×10⁻¹⁰ M, 1×10⁻¹¹ M, 1×10⁻¹² M, or more, and/or bind to a target with an affinity that is at least two-fold greater than its affinity for a nonspecific antigen.

As used herein, the term “immunologically challenged” refers to the exposure and immunological response of an animal to an antigen.

As used herein, the term “naïve animal” refers to an animal that has not been immunologically challenged with an antigen of interest.

As used herein, the term “natural repertoire of antigen receptors” refers to the repertoire of antigen receptors expressed naturally expressed in the immune cells of an animal.

As used herein, the term “synthetic antigen receptors” refers to the non-naturally occurring antigen receptors.

### II. Antigen Receptors

The methods of the invention are useful for identifying antigen receptors that bind specifically to an antigen of interest. Any cell surface antigen receptor that, when bound to an antigen, can elicit activation of a cell surface adhesion molecule can be employed in the methods of the invention.

In certain embodiments, the antigen receptor comprises the membrane-bound antibody component of a B-cell receptor. Any antibody capable of being displayed on the surface of a cell and, when bound to an antigen, can elicit activation of a cell surface adhesion molecule can be employed in the methods of the invention. Antibodies can be from any animal that produces an antibody including, but not limited to, rodent, lagomorph, avian, camelid, shark, or primate (e.g., human). Antibodies can be of any isotype including, without limitation, IgA, IgE, IgM, IgG, and IgD. Antibodies can be can be artificial, naturally derived, or a combination thereof. In a preferred embodiment, the antigen receptor is a fully human antibody.

In certain embodiments, the antigen receptor comprises the αβ or βγ chains of a T-cell receptor. Any T-cell receptor capable of being displayed on the surface of a cell and, when bound to an antigen, can elicit activation of a cell surface adhesion molecule can be employed in the methods of the invention. T-cell receptors can be from any animal that possess T-cells including, but not limited to, rodent, lagomorph, avian, camelid, shark, or primate (e.g., human). T-cell receptors can be can be artificial, naturally derived, or a combination thereof. In a preferred embodiment, the antigen receptor is a fully human T-cell receptor.

In other embodiments, the antigen receptor is a chimeric molecule comprising at least a portion of an antigen receptor (B-cell and/or T-cell receptor) linked (either chemically or genetically) to a heterologous binding molecule. Suitable heterologous binding molecule include, without limitation, antibody fragments or derivatives, and alternative binding scaffold molecules.

Suitable antibody fragments or derivatives include, without limitation, single domain antibodies (see, for example, Ward et al., Nature 341:544 (1989), which is incorporated by reference herein in its entirety), Fab fragments, single chain antibodies (see, for example, Bird et al. (1988) Science 242:423-426, is incorporated by reference herein in its entirety), unibodies (see, for example, WO2007/059782, which is incorporated by reference herein in its entirety), heavy chain only antibodies, and nanobodies (see, for example, U.S. Pat. No. 5,759,808, which is incorporated by reference herein in its entirety).

Suitable alternative binding scaffold molecules include, without limitation, fibronectin domains (see, for example, Koide et al. (2007), Methods Mol. Biol. 352: 95-109, which is incorporated by reference herein in its entirety), DARPin (see, for example, Stumpp et al. (2008) Drug Discov. Today 13 (15-16): 695-701, which is incorporated by reference herein in its entirety), Z domains of protein A (see, Nygren et al. (2008) FEBS J. 275 (11): 2668-76, which is incorporated by reference herein in its entirety), Lipocalins (see, for example, Skerra et al. (2008) FEBS J. 275 (11): 2677-83, which is incorporated by reference herein in its entirety), Affilins (see, for example, Ebersbach et al. (2007) J. Mol. Biol. 372 (1): 172-85, which is incorporated by reference herein in its entirety), Affitins (see, for example, Krehenbrink et al. (2008). J. Mol. Biol. 383 (5): 1058-68, which is incorporated by reference herein in its entirety), Avimers (see, for example, Silverman et al. (2005) Nat. Biotechnol. 23 (12): 1556-61 , which is incorporated by reference herein in its entirety), Fynomers, (see, for example, Grabulovski et al. (2007) J Biol Chem 282 (5): 3196-3204 , which is incorporated by reference herein in its entirety), and Kunitz domain peptides (see, for example, Nixon et al. (2006) Curr Opin Drug Discov Devel 9 (2): 261-8, which is incorporated by reference herein in its entirety).

In certain embodiments, the invention employs nucleic acid libraries encoding a plurality of unique antigen receptors (e.g., B-cell receptors). The libraries can comprise natural repertoires of antigen receptors (e.g., a portion of the antigen receptors normally expressed in one or more vertebrate animal) and/or a plurality synthetic antigen receptors (i.e., antigen receptors not normally expressed a vertebrate animal). In a preferred embodiment, the libraries comprise a repertoire of fully human antigen receptors from isolated from the B-cells of one or more human subjects. Methods for the generation of antigen receptor libraries are well known in the art (see for example, the methods set forth in U.S. Pat. No. 6,291,159, which is incorporated by reference herein in its entirety).

### III. Antigen Receptor-Expressing Cells

Any cell that expresses cell-surface antigen receptor is suitable for use in the methods of the invention provided that binding of the cell-surface antigen receptor to a cognate antigen results in activation of an adhesion molecule on the surface of that cell. Cells suitable for use in the present invention include, without limitation, normal primary cells (e.g., isolated B-cells), tumor cells (e.g., lymphoma cells), hybridomas, or immortalized primary cells.

In certain embodiments, the antigen receptor expressing cells are vertebrate cells including, without limitation, primate (e.g., human), rodent, lagomorph, chicken and camelid cells. The vertebrate cells can be derived from any vertebrate organ, but are preferably derived from leukocytes (e.g., lymphocytes, neutrophils, eosinophils, basophils, monocytes, macrophages, and dendritic cells). In a particular embodiment, the vertebrate cells are lymphocytes of the B cell lineage. These cells are particularly suitable for expression of B-cell receptors because they express antibody-specific chaperone proteins and accessory molecules that mediate cell surface expression of the antibody component of the B-cell receptor. B-cells can isolated from a naïve animal (i.e. an animal that has not been challenged with an antigen of interest) or from a animal that has been previously challenged with an antigen of interest. In a preferred embodiment, B-cells are isolated from a human that has been previously challenged with an antigen of interest. This method is particularly useful because it allows for the identification of fully human antigen-specific antibodies.

In certain embodiments, the antigen receptor-expressing cells are hybridoma cells formed by the fusion of a leukocyte (e.g. a B-cell) to myeloma cell. Methods for producing hybridoma cells are well known in the art. See, for example, WO 90/13660 which describes the production of human hybridoma cells by fusing human B-cells with heteromyeloma cells, and which is hereby incorporated by reference in its entirety.

In certain embodiments, the antigen receptor expressing cells are primary cells that have been immortalized. Any method of cell immortalization can be used in the methods of the invention including, without limitation, Epstein-Barr Virus (EBV) transformation (see, e.g., WO 04/76677, which is incorporated herein by reference in its entirety), or heterologous expression of genes that inhibit apoptosis, such as bcl-6 and/or bcl-xl (see, e.g., Kwakkenbos, et al., (2010) Nature Medicine 16(1) 123-129, which is incorporated herein by reference in its entirety). Such immortalization is especially useful in situations where cells undergo apoptosis in response to antigen binding, as is the case with naïve B-cells.

In certain embodiments, the antigen receptor expressing cells express an endogenous antigen receptor (i.e., an antigen receptor normally expressed by that cell). In other embodiments, cells express one or more heterologous antigen receptor(s) (i.e., an antigen receptor not normally expressed by that cell). Methods for achieving such heterologous expression are routine in the art (see for example the methods set forth in, for example, Antibody Engineering: Methods and Protocols. Methods in Molecular Biology Volume 248, (B. K. C. Lo, Ed) Humana Press, 2004 (ISBN: 1-58829-092-1), which is hereby incorporated by reference in its entirety).

Antigen receptor expressing cells that do not naturally express all the cellular components necessary to elicit adhesion molecule activation in response to antigen binding to a cell surface antigen receptor (e.g, intracellular signaling molecules, components of an antigen receptor, and adhesion molecules) can also be used in the methods of the invention. In such cases, it is necessary to heterologously express the missing proteins in the cells to facilitate antigen-mediated adhesion molecule activation. In one particular embodiment, the antigen receptor expressing cells heterologously express a component of an antigen receptor. Exemplary antigen receptor components include, without limitation, the Igα and/or Igβ components of a B-cell receptor.

In certain embodiments, a population of antigen receptor-expressing cells is employed, wherein the population expresses a diverse library of antigen receptors (e.g., B-cell receptors). The population of antigen receptor-expressing cells can be isolated from a vertebrate animal. Alternatively, the population of antigen receptor-expressing cells can be generated in vitro by the introduction of nucleic acid (e.g., in the form of expression vectors) encoding a diverse library of heterologous antigen receptors (e.g., B-cell receptors). The diverse library can comprise a natural repertoire of antigen receptors (e.g., a portion of the antigen receptors normally expressed in one or more vertebrate animal) and/or a plurality of unique, synthetic antigen receptors (i.e., antigen receptors not normally expressed a vertebrate animal). In a preferred embodiment, the population of antigen receptor-expressing cells expresses a diverse library of fully human antigen receptors. Methods for the generation of antigen receptor libraries are well known in the art (see for example, the methods set forth in U.S. Pat. No. 6,291,159, which is incorporated by reference herein in its entirety).

### IV. Antigens and Antigen Presentation

Any antigen is suitable for use in the methods of the invention including without limitation, polypeptide, carbohydrate, lipid and small molecule antigens. However, the skilled artisan will appreciate that the method of presenting an antigen to an antigen receptor-expressing cell will vary depending upon the type of antigen receptor expressed.

In the case of cells expressing a B-cell receptor, an antigen can be contacted directly with an antigen receptor-expressing cell. The antigen can be added in soluble form to cell culture media or buffer containing antigen receptor-expressing cells. Additionally or alternatively, the antigen can be adhered to a substrate (e.g., an optical biosensor) upon which antigen receptor-expressing cells are placed.

In the case of cells expressing a T-cell receptor (αβ and/or βγ), it may be necessary to present antigens to the cells in the form of short peptides in complex with MHC molecules. Isolated MHC/peptide complexes can be added in soluble form to cell culture media containing antigen receptor-expressing cells. Additionally or alternatively, isolated MHC/peptide complexes can be adhered to a substrate (e.g., an optical biosensor) upon which antigen receptor-expressing cells are placed. Additionally or alternatively, antigens can be presented to T-cell receptor-expressing cells as MHC/peptide complexes on the surface of cells (e.g., dendritic cells). Methods of displaying peptide antigens to T-cell receptor-expressing cells are well known in the art (see for example WO1992007952, which is incorporated by reference herein in its entirety)

### V. Adhesion Molecules

Any cell surface expressed adhesion molecule that becomes activated upon the binding of an antigen to a cell-surface antigen receptor is suitable for measurement in the methods of the invention. Suitable adhesion molecules include without limitation, integrins, cadherins and selectins.

Integrin activation is particularly suitable for measurement in the methods of the invention. Integrin molecules on the surface of unstimulated immune cells exist in an inactive conformation and do not bind to extracellular matrix proteins. Binding of an antigen to the antigen receptor on immune cells leads to activation of the cell surface integrins such that they can now bind to extracellular matrix proteins (for a discussion of integrin activation see, e.g., Arana et al. (2008) J. Cell. Sci. 121(14); 2279-2286, which is incorporated by reference herein in its entirety). Exemplary integrins include Leukocyte Functional Antigen 1 (LFA-1) and Very Late Antigen 4 (VLA-4). Exemplary LFA-1 molecules include the human LFA-1 comprising the two subunits set forth in genbank accessions GI:167466217 and GI:188595677, and the mouse LFA-1 comprising the two subunits set forth in genbank accession GI:198786 and GI:111607447. Exemplary VLA-4 molecules include the human VLA-4 comprising the two subunits set forth in genbank accession GI:119631392 and GI:19743819, and the mouse VLA-4 comprising the two subunits set forth in genbank accession GI:114326554 and GI:45504394.

In certain embodiments, the adhesion molecule to be measured is an endogenous adhesion molecule (i.e., a molecule normally expressed in the antigen receptor-expressing cell being assayed). In other embodiments, the adhesion molecule to be measured, or a component thereof, is heterologously expressed in the antigen receptor-expressing cell being assayed.

### VI. Measurement of Adhesion Molecule Activation

Any method of measuring the activation of a cell-surface adhesion molecule upon the binding of an antigen to a cell-surface antigen receptor is suitable for use in the methods of the invention. In general the activation of a cell-surface adhesion molecule is detected by measuring the binding of an antigen receptor-expressing cell to one or more extracellular matrix proteins. Additionally or alternatively, activated cell-surface adhesion molecule can be detected using antibodies that bind to an adhesion molecule in the activated state but not in the inactive state.

In certain embodiments, the activation of a cell surface adhesion molecule is detected using a label-free binding assay. Such assays generally comprise immobilizing an extracellular matrix protein on a biosensor and measuring the binding of an antigen receptor-expressing cell to the biosensor in the presence and absence of an antigen. An increase in the binding of the antigen receptor-expressing cell to the biosensor in the presence of the antigen indicates that the antigen receptor-expressing cell binds to the antigen.

In a preferred embodiment, the activation of a cell surface adhesion molecule is detected using a colorimetric resonant reflectance optical biosensor. Suitable colorimetric resonant reflectance optical biosensors and methods for use thereof are disclosed in US patent application numbers 2004/0091397, 20100196925, 20100221847, 20100202923, 20070172894, 20100291575, which are hereby incorporated by reference in their entirety.

Any extracellular matrix protein that binds to an antigen-activated adhesion molecule can be used in a label-free binding assay. Suitable extracellular matrix proteins include, without limitation, Inter-Cellular Adhesion Molecule 1 (ICAM-1) and fibronectin. Exemplary ICAM-1 molecules include the human ICAM-1 set forth in genbank accessions GI:825682, and the mouse ICAM-1 set forth in genbank accession GI:124099. Exemplary fibronectin molecules include the human fibronectin set forth in genbank accession GI:300669710, and the mouse fibronectin set forth in genbank accession GI:1181242.

In a label-free binding assay, one or more extracellular matrix protein(s) are applied to the surface of a biosensor. The extracellular matrix proteins can be applied uniformly to the surface of a biosensor or can be arrayed in discrete spots. Extracellular matrix proteins can be applied to the biosensor alone or together with an antigen of interest.

In certain embodiments, the activation of a cell surface adhesion molecule is detected using a Fluorescence Activated Cell Sorter (FACS). Such assays generally comprise: contacting an antigen receptor-expressing cell with an antibody, wherein the antibody binds to a cell surface adhesion molecule in the activated state but not in the inactive state; and detecting the binding of the antibody to the antigen receptor-expressing cell in the presence and absence of an antigen using a FACS. The binding of the antibody to the antigen receptor-expressing cell in the presence of the antigen indicates that the antigen receptor-expressing cell bind to the antigen.

### VII. Isolation of Antigen-Specific Antigen Receptors

Once an antigen-receptor expressing cell that binds to an antigen of interest (i.e., an antigen-binding cell) has been identified, the amino acid sequence of the antigen receptor expressed by that cell is determined. Any art recognized means for determining the amino acid sequence of the antigen receptor can be employed in the methods of the invention. In general, antigen-binding cells are clonally isolated and the nucleic acid encoding the expressed antigen receptor is isolated and sequenced.

In certain embodiments, antigen-binding cells are clonally isolated from a population of cells using a cell-picker device. Suitable cell-picker devices include, without limitation, those set forth in US patent application number 20030179916, which is hereby incorporated by reference in its entirety.

In other embodiments, antigen-binding cells can be isolated from a population of cells by limiting dilution into multi-well plates.

In other embodiments, antigen-binding cells are isolated from a population of cells using a FACS machine and a sortable label that binds specifically to the antigen-binding cells. In some embodiments, the sortable label is a fluorescently labelled antibody that specifically binds to an activated cell surface adhesion molecule on antigen-binding cell. In other embodiments, the sortable label is a fluorescently labelled antigen of interest.

Additionally or alternatively, antigen-binding cells can be isolated from a population of non-antigen binding cells based on their ability to bind to a substrate comprising an extracellular matrix protein and/or an antigen of interest.

The nucleic acid encoding an expressed antigen receptor can be isolated from an antigen-binding cell using an art recognized means including, without limitation, polymerase chain reaction (PCR) amplification using nucleic acid primers specific for conserved regions of the antigen receptor gene. The isolated nucleic acid encoding an expressed antigen receptor can then be sequenced (and the encoded amino acid sequence deduced) using any art recognized methods.

Once the nucleic acid encoding of antigen receptor that specifically binds to an antigen of interest has been isolated, it can be heterologously expressed in vitro (e.g., in cells or in a cell-free expression system) or in vivo (e.g., in a transgenic animal).

### EXAMPLES

The present invention is further illustrated by the following examples which should not be construed as further limiting.

### Example 1

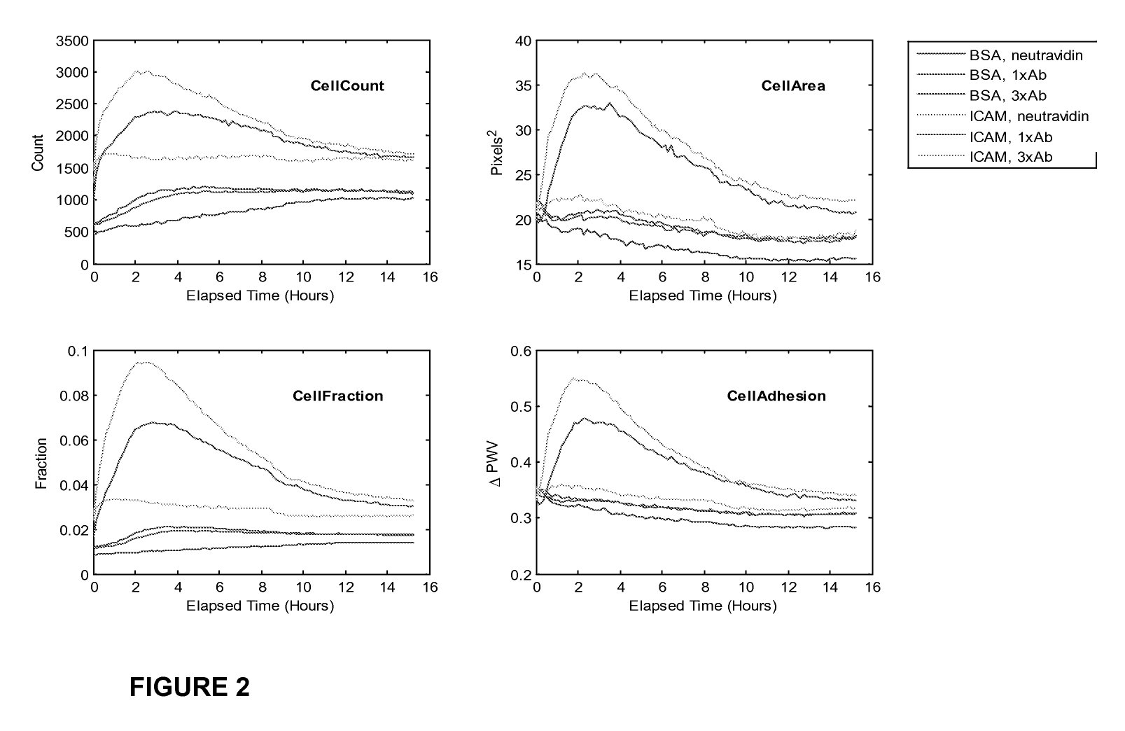

This example demonstrates that B-cell receptor-mediated activation of integrin LFA-1 on the surface of B-cells can be detected using a colorimetric resonant reflectance optical biosensor using a BIND™ scanner. A 96-well BIND™ TiO₂ biosensor plate was coated with Inter-Cellular Adhesion Molecule 1 (ICAM-1) protein at 1 μg/ml concentration at room temperature for 1 hour. The biosensor was also blocked with bovine serum albumin (BSA). B-cell lymphoma cells (line RL from ATCC) were then plated on the ICAM-1 coated BIND biosensor at 40,000 cells per well and grown in full growth medium. Biotinylated goat anti-human IgM antibody F(ab)₂ fragments were incubated with neutravidin for 30 min to 1 hour at room temperature to allow multimerization of the F(ab)₂ fragments. The multimerized IgM antibody fragments were then added to the B-cell lymphoma cell culture to cross-link the cell surface B-cell receptors. Binding of the cells to the biosensor was assessed by scanning the biosensor with the BIND™ scanner for 15 hours at room temperature at 3.75 pm resolution (sampling every 10 mins). The data set forth in FIG. 2 shows that the B-cell receptor crosslinking resulted in increased binding of the B-cells to the biosensor, and hence that B-cell receptor activation resulted in detectable activation of LFA-1 on the surface the cells.

We claim:

1. A method for identifying an antigen receptor that specifically binds to an antigen of interest, the method comprising:
(a) contacting a plurality of antigen receptor-expressing cells with the antigen;
(b) measuring the amount of activated adhesion molecules on the surface of the antigen receptor-expressing cells in the presence and absence of the antigen; and
(c) identifying from the plurality of antigen receptor-expressing cells an antigen receptor-expressing cell that specifically binds to the antigen, wherein an increase in the amount of activated adhesion molecules on the surface of an antigen receptor-expressing cell in the presence of the antigen relative to a suitable control is indicative of the binding of the antigen to the antigen receptor-expressing cell, thereby identifying an antigen receptor that specifically binds to an antigen of interest.

2. The method of claim 1, further comprising clonally isolating the antigen receptor-expressing cell identified in step (c).

3. The method of claim 1, further comprising determining the nucleic acid or amino acid sequence of at least a portion of the antigen receptor identified in step (c).

4. The method of any of the preceding claims, wherein the adhesion molecules are integrins.

5. The method of claim 4, wherein the integrin is a Leukocyte Functional Antigen 1 (LFA-1) or Very Late Antigen 4 (VLA-4) molecule.

6. The method of any of the preceding claims, wherein the amount of activated adhesion molecules is measured by measuring the binding of the antigen receptor-expressing cells to an extracellular matrix protein or to an antibody that binds to activated adhesion molecules but not to quiescent adhesion molecules.

7. The method of claim 5, wherein the extracellular matrix protein is an Inter-Cellular Adhesion Molecule 1 (ICAM-1) or a fibronectin molecule.

8. The method of claim 6 or 7, wherein the binding of the antigen receptor-expressing cells to the extracellular matrix protein or the antibody is measured using a label-free biosensor coated with the extracellular matrix protein or the antibody.

9. The method of claim 8, wherein the biosensor is a colorimetric resonant reflectance optical biosensor.

10. The method of any of the preceding claims, wherein the antigen receptor is a B-cell receptor.

11. The method of claim 10, wherein the B-cell receptor is a human B-cell receptor.

12. The method of any of the preceding claim, wherein the antigen receptor-expressing cells are B-cells or hybridoma cells.

13. The method of claim 12, wherein the B-cells are isolated from one or more naïve animals.

14. The method of claim 12, wherein the B-cells are isolated from one or more animals that have not been immunologically challenged with the antigen of interest.

15. The method of claim 13 or 14, wherein the animal is a human.

16. The method of claim 12, wherein the B-cells have been immortalized.

17. The method of claim 12, wherein the B-cells express endogenous antibodies.

18. The method of claim 12, wherein the B-cells express a library of heterologous antibodies.

19. The method of claim 18, wherein the library comprises a natural repertoire of unique antibodies.

20. The method of claim 19, wherein the library is a naïve antibody library.

21. The method of claim 18, wherein the library comprises human antibodies.

22. The method of claim 18, wherein the library comprises a plurality of unique synthetic antigen receptors.

23. The method of claim 12, wherein the B-cells express a library of unique chimeric antigen receptors, wherein each chimeric receptor comprises a portion of an antigen receptor linked to a heterologous binding molecule.

24. A method for producing an antigen receptor that specifically binds to an antigen of interest, the method comprising:
(a) identifying an antigen receptor according to the method of any one of the preceding claims; and,
(b) expressing the antigen receptor, or an antigen-binding portion thereof.

25. The method of claim 24, wherein the antigen receptor is an antibody.

26. The method of claim 25, further comprising determining the nucleic acid or amino acid sequence of at least one complementarity determining region (CDR) of the antibody.

27. The method of claim 26, further comprising grafting the at least one CDR into the framework of a heterologous antibody.
